(19) United States
(12) Patent Application Publication
(10) Pub. No.: US 2006/0263761 A1
(43) Pub. Date: Nov. 23, 2006

(54) Methods and compositions for beryllium-induced Disease

(76) Inventors: Andrew P. Fontenot, Denver, CO (US); Brian L. Kotzin, Westlake, CA (US); Lee S. Newman, Denver, CO (US); Lisa Maier, Denver, CO (US)

Correspondence Address: FAEGRE & BENSON LLP;PATENT DOCKETING, 2200 WELLS FARGO CENTER, 90 SOUTH 7TH STREET, MINNEAPOLIS, MN, 55402-3901, US

(21) Appl. No.: 11/373,581

(22) Filed: Mar. 10, 2006

Related U.S. Application Data
(60) Provisional application No. 60/660,622, filed on Mar. 11, 2005.

(51) Int. Cl. C12Q 1/00; G01N 33/567; G01N 33/20

(52) U.S. Cl. 435/4; 435/7.2; 436/80

(57) ABSTRACT

The present invention provides for methods for detection, diagnosis and prognosis of beryllium-induced disease. In one embodiment, the methods include exposing immune cells from subjects suspected of having beryllium-induced disease to beryllium and assessing the Th-1 cytokines produced. Other embodiments include the use of exposing immune cells from subjects suspected of having beryllium-induced disease to beryllium and assessing Th-1 cytokines produced and using these assessments to indicate the stage of progression of the disease. Therapeutic methods involve assessing the onset or progression of beryllium-induced disease before during and after exposure to a treatment for the disease.

### CROSS REFERENCE TO RELATED APPLICATIONS

This application claims the benefit under 35 U.S.C. §119(e) of provisional U.S. patent application Ser. No. 60/660,622 filed on Mar. 11, 2005. The aforementioned application is hereby incorporated by reference in its entirety for all purposes.

### FEDERALLY FUNDED RESEARCH

The studies disclosed herein were supported in part by grant PO1 ES11810 from the National Institute of Environmental Health Sciences, NIH. The U.S. government may have certain rights to practice the subject invention.

### FIELD

The present invention relates to methods and compositions for detection, diagnosis, progression and prognosis of disease. In one embodiment, the disease may be a non-infectious disease. In one embodiment, the disease may be beryllium-induced disease. In one embodiment, a method may include a non-invasive technique for exposing immune system cells from subjects suspected of having beryllium-induced disease to beryllium and measuring expression of cytokines. In one particular embodiment, the cytokines may -include Th-1 type cytokines (T helper-1 type inflammatory cytokines) such as IFN-γ (interferon gamma) and/or IL-2 (interleukin-2). In more particular embodiments, the number of beryllium-specific CD4+ T cells detected may be used to monitor disease progression from beryllium-sensitivity (BeS) to chronic beryllium disease (CBD). In another embodiment, the presence of or absence of Th-1 type cytokines may be measured.

### BACKGROUND

Beryllium sensitization occurs in individuals exposed to beryllium in the workplace, with greater than 1,000,000 U.S. workers having been exposed and thus at risk for its development. Beryllium-sensitized (BeS) individuals possess a beryllium-specific immune response, which is limited to blood and shows no evidence of lung disease. Only a subset of these individuals progress to chronic beryllium disease (CBD). Depending on the nature of the exposure and the genetic susceptibility of the individual, it is estimated that disease develops in 1-16% of exposed individuals.

CBD is characterized by granulomatous inflammation and the accumulation of beryllium-specific CD4+ T cells in the lung. Lung T cells are involved in the immunopathogenesis of disease and are composed of oligoclonal T cell expansions that recognize beryllium in an HLA-DP-restricted manner. Although the vast majority of beryllium-specific CD4+ T cells from CBD patients are compartmentalized to the lung, blood T cells proliferate in the presence of beryllium salts in culture. The immunologic mechanisms involved in the progression from beryllium sensitization to CBD remain poorly defined.

One standard assay for documenting the presence of a beryllium-specific immune response in blood is the beryllium lymphocyte proliferation test (BeLPT). This assay has been used for screening and diagnosis of beryllium sensitization in the workplace and is a required component of the US Department of Energy CBD prevention program. However, it has been criticized due to variability in test results. In addition, the BeLPT is not capable of distinguishing between BeS and CBD. Consequently, invasive tests are required such as bronchoscopy with bronchoalveolar lavage (BAL) and lung biopsy to confirm progression to CBD.

A need exists for noninvasive assays to detect beryllium sensitization and to differentiate stages of disease, particularly to monitor progression from BeS to CBD. Such an assay would be of great use to enhance patient care for beryllium-exposed individuals and avoid invasive pulmonary procedures.

### SUMMARY

The present invention provides methods and compositions for non-invasive detection, diagnosis, staging and prognosis of disease. In one embodiment, the disease may be a non-infectious disease. In one particular embodiment, the disease may be beryllium-induced disease. In particular embodiments, the methods can involve detection and/or measurement of Th-1 type cytokines, such as IFN-γ or IL-2 before during or after exposure to a metal. In accordance with this embodiment, the metal may be an alkali earth metal, transition metal or other metal. For example, the metal may include but is not limited to aluminum, antimony, arsenic, barium, beryllium, cadmium, chromium, cobalt, copper, iron, lithium, manganese, mercury, nickel, platinum, rhodium, rare earth metals, titanium, uranium, vanadium, welding, zinc, and zirconium. In one particular embodiment, the metal is beryllium. In another particular embodiment, a specialized assay may be used to measure cytokine production in activated CD4+ T cells (e.g. beryllium-activated cells) and compared to control-treated and/or untreated cells. In accordance with these embodiments, an assay to measure cytokine production in activated CD4+ T cells can be an ELISPOT assay.

### BRIEF DESCRIPTION OF THE DRAWINGS

The following drawings form part of the present specification and are included to further demonstrate certain exemplary embodiments of the present invention. The embodiments may be better understood by reference to one or more of these drawings in combination with the detailed description of specific embodiments presented herein.

FIG. 1. demonstrates an exemplary frequency of beryllium-specific T cells in blood. PBMCs were evaluated using ELISPOT analysis for IFN-γ (A) and IL-2 (B) after beryllium exposure in culture. Data are expressed as the mean SFU, and median values are indicated with solid lines. Immunosuppressant-treated CBD subjects are indicated by open triangles. Above the dotted line represents a positive cytokine response.

FIG. 2. demonstrates exemplary receiver operator characteristic (ROC) curves for IFN-γ and IL-2 SFU in the detection of beryllium sensitization (BeS and CBD) following exposure to 1×10⁻⁴ M BeSO₄. The area under the curve (AUC) is shown. The chosen cutoff value for each ROC curve is also shown.

FIG. 3. demonstrates exemplary beryllium-induced proliferative responses of PBMCs. The response to 1×10⁻⁴ M and 1×10⁻⁵ M BeSO₄ in culture is depicted as stimulation index (SI). An SI indicative of a positive response is currently defined as ≧2.5 (dotted line). Median values are shown and indicated with a horizontal line.

FIG. 4. demonstrates exemplary intracellular expression of Th1-type cytokines in PBMCs. A. Representative experiment is shown for BeSO₄-stimulated PBMCs from a CBD patient. B. Analyses of IFN-γ versus IL-2 are shown for CD4+ T cells from two representative CBD patients following BeSO₄ exposure. C. Frequency of IL-2- and IFN-γ-expressing, beryllium specific CD4+ T cells in blood (n=11) is shown, and median value is shown.

### DESCRIPTION OF ILLUSTRATIVE EMBODIMENTS

### DEFINITIONS

Terms that are not otherwise defined herein are used in accordance with their plain and ordinary meaning.

As used herein, “a” or “an” may mean one or more than one of an item.

As used herein, “about” means within plus or minus ten (10) percent of a value. For example, “about 100” refers to any number between 90 and 110.

The abbreviations used herein are as listed below.

BAL: Bronchoalveolar lavage

BeLPT: Beryllium lymphocyte proliferation test

BeS: Beryllium-sensitized

BeSO4: Beryllium sulfate

CBD: Chronic beryllium disease

CPM: Counts per minute

DLCO: Diffusing capacity for carbon monoxide

PBMC: Peripheral blood mononuclear cells

PHA: Phytohemagglutinin

ROC: Receiver Operator Characteristic

SEB: Staphylococcal enterotoxin B

SFU: Spot-forming units

SI: Stimulation index

### DETAILED DESCRIPTION

In the following description, several specific details are presented such as examples of specific methods, components, and processes in order to provide a thorough understanding of various embodiments. It will be obvious to one skilled in the art that these specific details need not be employed to practice the various embodiments. In other cases, some well-known components or methods will not be described in detail in order to alleviate unnecessary obscuring of various embodiments presented forthwith.

In one embodiment of the present invention, methods and compositions are provided for non-invasive detection, diagnosis, progression and prognosis of metal-induced disease or a non-infectious disease such as a beryllium-induced disease, an aluminum-induced disease or a nickel-induced disease. In one embodiment, the methods may involve detection and/or measurement of a specific T-cell population such as CD4+ T-cells. In another embodiment, the methods may involve detection and/or measurement of Th-1 type cytokines. In accordance with this embodiment, the methods may involve detection and/or measurement of presence of or levels of T-cell expressed cytokines such as Th-1 type cytokines (e.g. IFN-γ or IL-2) after exposure to a non-peptidetic agent such as a chemical or a metal ion. For example, a chemical agent may be a carcinogen such as asbestos, benzene, DDT, formaldehyde, mustard gas etc. In another example, a metal can be an alkali earth or transition metal. For example, a metal may include but are not limited to aluminum, antimony, arsenic, barium, beryllium, cadmium, chromium, cobalt, copper, iron, lithium, manganese, mercury, nickel, platinum, rhodium, rare earth metals, titanium, uranium, vanadium, welding, zinc, zirconium.

In a more particular embodiment, the methods may involve detection and/or measurement of Th-1 type cytokines such as IFN-γ or IL-2 after exposure to the metal, beryllium. In accordance with this embodiment, a specialized assay may be used to measure cytokine production in beryllium-activated CD4+ T cells and compared to control-treated and/or untreated cells. In one particular embodiment, an assay may be used to measure cytokine production in beryllium-activated CD4+ T cells and compared to control-treated and/or untreated cells. In a more particular embodiment, an ELISspot assay may be used to measure cytokine production in beryllium-activated CD4+ T cells and compared to control-treated and/or untreated cells.

In one particular embodiment, a specialized assay designed to rapidly measure cytokine production from cells may be used to assess Th-1 type cytokines such as IFN-γ or IL-2 produced by metal-activated CD4+ T cells. In one particular embodiment, an assay may be used to assess Th-1 type cytokines such as IFN-γ or IL-2. A specific assay for cytokine production such as an Elispot assay are advantageous over current proliferation assays for beryllium induced disease such as the BeLPT test as a diagnostic tool. Some of these advantages include: [1] determination of an accurate reflection of beryllium-specific cells in blood, [2] the ability to track these cells over time, [3] the ability to detect cells which are capable of secreting Th1-type cytokines but are no longer able to proliferate in response to beryllium, [4] shorter duration of assay (24 hours versus 6 days), and [5] lack of radioactivity. In contrast, BeLPT is directed toward determining cell proliferation in the presence of beryllium.

One aspect of the present application includes experiments that demonstrate chronic beryllium disease (CBD) patients possess a greater number of beryllium-specific T cells in blood compared to beryllium-sensitized (BeS) subjects. In accordance with this aspect, one embodiment includes distinguishing stages or progression of beryllium disease by assessing the number of beryllium-specific CD4⁺ T cells in blood circulation. In a more particular embodiment, one or more samples such as a sample of peripheral blood, of enriched white cell fraction of blood or of bronchoalveolar lavage may be obtained from a subject having or suspected of developing beryllium disease and the number of beryllium-specific CD4⁺ T cells may be measured in the sample. In accordance with this embodiment, the concentration or number of beryllium-specific CD4⁺ T cells found in the blood may be correlated with the presence or stage of beryllium disease.

In one embodiment, the number of beryllium-specific CD4⁺ T cells may be monitored by exposing immune system cells such as CD4⁺ T cells from a subject having or suspected of developing beryllium disease to beryllium and then evaluating the concentration of cytokine produced from such an exposure. For example, Th-1 type cytokines, such as IFN-γ or IL-2 produced from the exposed immune cells may be measured. In accordance with these embodiments, an assay such as the ELISpot assay may be used to assess the level of cells producing cytokines (e.g. IFN-γ and/or IL-2) in a subject at a given time and this may be used to assess the onset or stage of beryllium-induced disease in the subject.

In another embodiment, transition from BeS to CBD may be associated with an increase in the frequency of beryllium-specific T cells in blood, indicating that alterations in the number of antigen-specific T cells in blood may be useful in predicting disease progression. The skilled artisan will realize that the disclosed methods are not limited to beryllium-induced disease but rather may be used in a variety of known non-infectious diseases for example, autoimmune diseases or other metal- or chemical-induced diseases, in particular those with an inaccessible target organ. In addition, the skilled artisan will realize that the disclosed methods may include applications to other metal-induced diseases for example copper, aluminum, and nickel-induced diseases.

In another embodiment, any of the methods disclosed herein may be combined with other known metal-disease assessment tests. For example, the methods disclosed herein may be combined with measuring the presence of beryllium in a given test sample (Chiarappa-Zucca et. al. (2004) Measurement of Beryllium in Biological Samples by Accelerator Mass Spectrometry: Applications for Studying Chronic Beryllium Disease, Chem Res. Toxicol. 17:1614-1620 incorporated by reference herein in its entirety). In another example, the methods disclosed herein may be combined with any test assessing the response to a therapeutic agent administered to a subject having a metal-induced disease in order to evaluate the progression of the disease in the subject and/or the response of the subject to the agent. In accordance with this embodiment, a therapeutic treatment may be altered and/or additional therapeutic treatments may be added according to these assessments.

Marker Genes

In certain aspects of the present invention, specific cells may be tagged with specific genetic markers to provide information about the fate of the tagged cells. Therefore, the present invention also provides recombinant candidate screening and selection methods which are based upon whole cell assays and which, preferably, employ a reporter gene that confers on its recombinant hosts a readily detectable phenotype that emerges only under conditions where a general DNA promoter positioned upstream of the reporter gene is functional. Generally, reporter genes encode a polypeptide (marker protein) not otherwise produced by the host cell which is detectable by analysis of the cell culture, e.g., by fluorometric, radioisotopic or spectrophotometric analysis of the cell culture.

In other aspects of the present invention, a genetic marker is provided which is detectable by standard genetic analysis techniques, such as DNA amplification by PCR™ or hybridization using fluorometric, radioisotopic or spectrophotometric probes.

Screening

Exemplary enzymes include esterases, phosphatases, proteases (tissue plasminogen activator or urokinase) and other enzymes capable of being detected by their activity, as will be known to those skilled in the art.

Other particular examples are the enzyme chloramphenicol acetyltransferase (CAT) which may be employed with a radiolabeled substrate, firefly and bacterial luciferase, and the bacterial enzymes β-galactosidase and β-glucuronidase. Other marker genes within this class are well known to those of skill in the art, and are suitable for use in the present invention.

Protein Purification

Further aspects of the present invention concern the purification, and in particular embodiments, the substantial purification, of a protein or peptide. The term “purified protein or peptide” as used herein, is intended to refer to a composition, isolatable from other components, wherein the protein or peptide is purified to any degree relative to its naturally-obtainable state, e.g., relative to its purity within a cell extract. A purified protein or peptide therefore also refers to a protein or peptide, free from the environment in which it may naturally occur.

Generally, “purified” will refer to a protein or peptide composition which has been subjected to fractionation to remove various other components, and which composition substantially retains its expressed biological activity. Where the term “substantially purified” is used, this will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number”. The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in the most purified state. Indeed, it is contemplated that less substantially purified products will have utility in certain embodiments. Partial purification may be accomplished by using fewer purification steps in combination, or by utilizing different forms of the same general purification scheme. Methods exhibiting a lower degree of relative purification may have advantages in total recovery of protein product, or in maintaining the activity of an expressed protein.

It is known that the migration of a polypeptide may vary, sometimes significantly, with different conditions of SDS/PAGE. It will therefore be appreciated that under differing electrophoresis conditions, the apparent molecular weights of purified or partially purified expression products may vary.

Antibody Production

Means for preparing and characterizing antibodies are well known in the art (See, e.g., Antibodies: A Laboratory Manual, Cold Spring Harbor Laboratory, 1988; incorporated herein by reference).

Methods for generating polyclonal antibodies are well known in the art. Briefly, a polyclonal antibody is prepared by immunizing an animal with an immunogenic composition and collecting antisera from that immunized animal. A wide range of animal species may be used for the production of antisera. Typically the animal used for production of anti-antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin may also be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As is also well known in the art, the immunogenicity of a particular immunogen composition may be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes may be used to administer the immunogen (subcutaneous, intramuscular, intradermal, intravenous and intraperitoneal). The production of polyclonal antibodies may be monitored by sampling blood of the immunized animal at various points following immunization. A second, booster injection, may also be given. The process of boosting and titering is repeated until a suitable titer is achieved. When a desired level of immunogenicity is obtained, the immunized animal may be bled and the serum isolated and stored, and/or the animal may be used to generate MAbs. For production of rabbit polyclonal antibodies, the animal may be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody or a peptide bound to a solid matrix.

Monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner effective to stimulate antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages, but mice are preferred, with the BALB/c mouse being most preferred as this is most routinely used and generally gives a higher percentage of stable fusions.

The animals are injected with antigen as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), may be selected for use in the MAb generating protocol. Any methods known in the art for producing and selecting MAbs may be used herein.

In accordance with embodiments of the present invention, fragments of the monoclonal antibody of the invention may be obtained from the monoclonal antibody produced herein, by methods which include digestion with enzymes such as pepsin or papain and/or cleavage of disulfide bonds by chemical reduction. Alternatively, monoclonal antibody fragments encompassed by the present invention may be synthesized using an automated peptide synthesizer.

Immunodetection Assays

Methods

In still further embodiments, the present invention concerns immunodetection methods for binding, purifying, removing, quantifying or otherwise generally detecting biological components. The encoded proteins or peptides of the present invention may be employed to detect antibodies having reactivity therewith, or, alternatively, antibodies prepared in accordance with the present invention, may be employed to detect the encoded proteins or peptides. The steps of various useful immunodetection methods have been described in the scientific literature, such as, e.g., Nakamura et al. (1987).

In general, the immunobinding methods include obtaining a sample suspected of containing a protein, peptide or antibody, and contacting the sample with an antibody or protein or peptide in accordance with the embodiments of the present invention, as the case may be, under conditions effective to allow the formation of immunocomplexes.

The immunobinding methods include methods for detecting or quantifying the amount of a reactive component in a sample, which methods require the detection or quantitation of any immunecomplexes formed during the binding process. Here, one would obtain a sample suspected of containing a cytokine and contact the sample with an antibody or encoded protein or peptide, as the case may be, and then detect or quantify the amount of immunecomplexes formed under the specific conditions.

In terms of antigen detection, the biological sample analyzed may be any sample, such as a tissue section or specimen, a homogenized tissue extract, an isolated cell, a cell membrane preparation, separated or purified forms of any of the above protein-containing compositions or even any biological fluid. Various embodiments include bone marrow aspirate, bone marrow biopsy, lymph node aspirate, lymph node biopsy, spleen tissue, fine needle aspirate, skin biopsy or organ tissue biopsy. Other embodiments include samples where the body fluid is peripheral blood, lymph fluid, ascites, serous fluid, pleural effusion, sputum, cerebrospinal fluid, lacrimal fluid, stool or urine.

Contacting the chosen biological sample with the protein, peptide or antibody under conditions effective and for a period of time sufficient to allow the formation of immunecomplexes (primary immunecomplexes) is generally a matter of simply adding the composition to the sample and incubating the mixture for a period of time long enough for the antibodies to form immunecomplexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immunecomplexes to be detected.

The encoded protein, peptide or corresponding antibody employed in the detection may itself be linked to a detectable label, wherein one would then simply detect this label, thereby allowing the amount of the primary immunecomplexes in the composition to be determined.

Alternatively, the first added component that becomes bound within the primary immunecomplexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immunecomplexes are contacted with the labeled, secondary binding ligand, or antibody, under conditions effective and for a period of time sufficient to allow the formation of secondary immunecomplexes. The secondary immunecomplexes are then generally washed to remove any non-specifically bound labeled secondary antibodies or ligands, and the remaining label in the secondary immunecomplexes is then detected.

Further methods include the detection of primary immunecomplexes by a two step approach. A second binding ligand, such as an antibody, that has binding affinity for the encoded protein, peptide or corresponding antibody is used to form secondary immunecomplexes, as described above. After washing, the secondary immunecomplexes are contacted with a third binding ligand or antibody that has binding affinity for the second antibody, again under conditions effective and for a period of time sufficient to allow the formation of immunecomplexes (tertiary immunecomplexes). The third ligand or antibody is linked to a detectable label, allowing detection of the tertiary immunecomplexes thus formed. This system may provide for signal amplification if this is desired.

The immunodetection methods of the present invention may be of utility in the diagnosis of various diseases or disease states. A biological or clinical sample suspected of containing either the encoded protein or peptide or corresponding antibody is used. However, these embodiments also have applications to non-clinical samples, such as in the titering of antigen or antibody samples, in the selection of hybridomas, and the like.

In the clinical diagnosis or monitoring of patients, the detection of an antigen encoded by a disease state marker nucleic acid, or an increase in the levels of such an antigen, in comparison to the levels in a corresponding biological sample from a normal subject is indicative of a patient with the disease. The basis for such diagnostic methods lies, in part, with the finding that the nucleic markers identified in the present invention are differentially expressed in tissue samples from individuals with the disease.

Those of skill in the art are very familiar with differentiating between significant expression of a biomarker, which represents a positive identification, and low level or background expression of a biomarker. Indeed, background expression levels are often used to form a “cut-off” above which increased staining will be scored as significant or positive. Significant expression may be represented by high levels of antigens in tissues or within body fluids, or alternatively, by a high proportion of cells from within a tissue that each give a positive signal.

Immunohistochemistry

The antibodies of the present invention may be used in conjunction with both fresh-frozen and formalin-fixed, paraffin-embedded tissue blocks prepared by immunohistochemistry (IHC). Any IHC method well known in the art may be used such as those described in Diagnostic Immunopathology, 2nd edition. edited by, Robert B. Colvin, Atul K. Bhan and Robert T. McCluskey. Raven Press, New York., 1995, (incorporated herein by reference).

ELISA

As noted, it is contemplated that the encoded proteins or peptides of the invention will find utility as immunogens, e.g., in connection with vaccine development, in immunohistochemistry and in ELISA assays. One evident utility of the encoded antigens and corresponding antibodies is in immunoassays for the detection of disease marker proteins, as needed in diagnosis and prognostic monitoring.

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like may also be used.

In one exemplary ELISA, antibodies binding to the proteins of the invention are immobilized onto a selected surface exhibiting protein affinity, such as a well in a polystyrene microtiter plate. Then, a test composition suspected of containing the diseased cells, such as a clinical sample, is added to the wells. After binding and washing to remove non-specifically bound immunecomplexes, the bound antigen may be detected. Detection is generally achieved by the addition of a second antibody specific for the target protein, that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the disease marker antigen are immobilized onto the well surface and then contacted with the antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Another ELISA in which the proteins or peptides are immobilized, involves the use of antibody competition in the detection. In this ELISA, labeled antibodies are added to the wells, allowed to bind to the marker protein, and detected by means of their label. The amount of marker antigen in an unknown sample is then determined by mixing the sample with the labeled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immunecomplexes. These are described as follows:

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These may include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control human prostate, bladder or breast cancer and/or clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immunecomplex with a urease, glucose oxidase, alkaline phosphatase or hydrogen peroxidase-conjugated antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

Kits

In still further embodiments, the present invention concerns kits for use with the methods described above. In one embodiment, an immunodetection kit is contemplated. In another embodiment, a kit for analysis of a sample from a subject having or suspected of developing a metal or chemically-induced disease is contemplated. In a more particular embodiment, a kit for analysis of a sample from a subject having or suspected of developing beryllium-induced disease is contemplated. In accordance with this embodiment, the kit may be used to assess the onset or the progression of the disease.

In accordance with an immunodetection kit, the following may be needed. As an encoded proteins or peptides may be employed to detect antibodies and the corresponding antibodies may be employed to detect encoded proteins or peptides, either or both of such components may be provided in the kit. The immunodetection kits will thus comprise, in suitable container means, an encoded protein or peptide, or a first antibody that binds to an encoded protein or peptide, and an immunodetection reagent.

In certain embodiments, the encoded protein or peptide, or the first antibody that binds to the encoded protein or peptide, may be bound to a solid support, such as a column matrix or well of a microtiter plate.

The immunodetection reagents of the kit may take any one of a variety of forms, including those detectable labels that are associated with or linked to the given antibody or antigen, and detectable labels that are associated with or attached to a secondary binding ligand. Exemplary secondary ligands are those secondary antibodies that have binding affinity for the first antibody or antigen, and secondary antibodies that have binding affinity for a human antibody.

Further suitable immunodetection reagents for use in the present kits include the two-component reagent that comprises a secondary antibody that has binding affinity for the first antibody or antigen, along with a third antibody that has binding affinity for the second antibody, the third antibody being linked to a detectable label.

The kits may further comprise a suitably aliquoted composition of the encoded protein or polypeptide antigen, whether labeled or unlabeled, as may be used to prepare a standard curve for a detection assay.

The kits may contain antibody-label conjugates either in fully conjugated form, in the form of intermediates, or as separate moieties to be conjugated by the user of the kit. The components of the kits may be packaged either in aqueous media or in lyophilized form.

The container means of any of the kits will generally include at least one vial, test tube, flask, bottle, syringe or other container means, into which the testing agent, the antibody or antigen may be placed, and preferably, suitably aliquoted. Where a second or third binding ligand or additional component is provided, the kit will also generally contain a second, third or other additional container into which this ligand or component may be placed. The kits of the present invention will also typically include a means for containing the antibody, antigen, and any other reagent containers in close confinement for commercial sale. Such containers may include injection or blow-molded plastic containers into which the desired vials are retained.

Single-Chain Antibodies

In yet another embodiment, one gene may comprise a single-chain antibody. Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule.

Single-chain antibodies can be synthesized by a cell, targeted to particular cellular compartments, and used to interfere in a highly specific manner with cell growth and metabolism. Recently, single-chain antibodies were utilized for the phenotypic knockout of growth-factor receptors, the functional inactivation of p21ras, and the inhibition of HIV-1 replication. Intracellular antibodies offer a simple and effective alternative to other forms of gene inactivation, as well as demonstrate a clear potential as reagents for cancer therapy and for the control of other non-infectious diseases as well as infectious diseases. Single-chain antigen-binding proteins also represent potentially unique molecules for targeted delivery of drugs, toxins, or radionuclides to a tumor site, and show increased accessibility to tumor cells in vivo (Yokoda et al., 1992).

The embodiments are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### EXAMPLES

### Example 1

### Detection of Th1-Type Cytokine Secretion by CD4+ T Cells in BeS and CBD Subjects

In one exemplary method beryllium-specific proliferative responses were analyzed. In accordance with this method, Th1-type cytokine secretion by CD4+ T cells in the blood of BeS and CBD subjects were assessed. Both patient groups when compared to healthy controls were associated with markedly elevated numbers of IFN-γ and IL-2-secreting T cells specific for beryllium. Although no difference in proliferative response was seen in BeS and CBD subjects, a higher frequency of antigen-specific, cytokine-secreting T cells in CBD than in BeS subjects was observed. This indicates that the number of circulating beryllium-specific, cytokine-secreting T cells increases as disease progresses, and may be of use to separate the stages of beryllium-induced disease. The assessment of circulating beryllium-specific, cytokine-secreting T cells may be used to monitor the progression from BeS to CBD. In addition, assessment of circulating beryllium-specific, cytokine-secreting T cells in a subject may be used to monitor a treatment of a subject having beryllium disease in order to assess the efficacy of the treatment. For example, a treatment for beryllium disease may include but is not limited to corticosteroids, methotrexate and azathioprine.

Subjects

In one exemplary method, thirty-three patients with a diagnosis of CBD and 18 patients with a diagnosis of BeS were enrolled in a study. The diagnosis of CBD was established by using previously denned criteria, including a history of beryllium exposure, presence of an abnormal blood BeLPT on 2 separate occasions and/or BAL BeLPT on 1 occasion, and the presence of granulomatous inflammation and/or mononuclear cell infiltration on lung biopsy. The diagnosis of BeS was established on the basis of a history of beryllium exposure, an abnormal blood BeLPT on 2 occasions, and the absence of granulomatous inflammation or other abnormalities on lung biopsy. In one example, BeS subjects underwent bronchoscopy with BAL and transbronchial biopsy to determine whether disease progression had occurred. Also, enrolled were some BeS subjects and subjects with CBD who repeatedly had normal or borderline blood BeLPT to evaluate the utility of an ELISPOT assay on this patient population. A total of 12 healthy nonberyllium-exposed control subjects were also enrolled.

The demographics of the BeS patients and patients with CBD are shown in Table 1. Active smokers were excluded from enrollment. No difference in years since diagnosis was observed between BeS subjects and subjects with CBD, regardless of the presence or absence of beryllium-induced proliferation of blood cells. Seven patients with CBD were treated with corticosteroids, and 4 received methotrexate. Indications for treatment include severe disabling symptoms, worsening pulmonary function and/or exercise physiology, and evidence of cor pulmonale. Despite the presence of granulomatous inflammation and a T-cell alveolitis in the patients with CBD, no difference in pulmonary or exercise physiology was observed, except in certain cases a decreased diffusing capacity for carbon monoxide in the CBD compared with the BeS group (median, 86; range, 51-121; vs median, 96; range, 73-117; P=0.04).

Exemplary Methods

Lymphocyte Proliferation Assay

In one exemplary method, proliferation assays were performed using PBMCs (2.5×10⁵ cells/well) cultured for 4-6 days in complete culture media containing RPMI 1640 supplemented with 10% heat-inactivated human serum (Gemini Bio-Products, Woodland Calif.) with the following stimulants: medium, 2.5 μg/ml phytohemagglutinin (PHA), 1×10⁻⁴ M or 1×10⁻⁵ M BeSO₄ (example of one beryllium composition). The wells were pulsed with 1 μCi of [³H] thymidine for 18 hours, and incorporation of radioactivity was determined by β-emission spectroscopy. Proliferation assays were performed in quadruplicate. The data are presented as stimulation index (SI) with a positive response defined as SI≦2.5.12. Any other means known in the art for assessing proliferation of cells may be used.

Immunofluoresence Staining and Analysis of Intracellular Cytokine Expression

In one exemplary method for stimulation of cytokine expression, 1×10⁶ PBMCs and 5×10⁵ BAL cells were exposed to either medium alone, 10 ng/ml staphylococcal enterotoxin B (SEB), or 1×10⁻⁴ M BeSO₄ for 6 hours with 10 μg/ml brefeldin A (an example of one beryllium composition) added after the first hour of stimulation. Cells were stained with mAbs directed against CD4 and CD8 (BD Biosciences Pharmingen) followed by fixation, permeabilization, and staining with mAbs directed against IFN-γ and/or IL-2 (Caltag, Burlingame, Calif.). The lymphocyte population was identified using forward and 90° light scatter patterns, and fluorescence intensity was analyzed using a FACScaliber cytometer (Becton Dickinson) as previously described but any means known in the art may be used to identify this population.

Analysis of IFN-γ and IL-2 Production by ELISPOT Assay

In one exemplary method, ELISPOT assays were performed using plates (ImmunoSpot M200, BD Biosciences Pharmingen) that were coated with, IFN-γ or IL-2 capture mAb (BD Biosciences Pharmingen) overnight and blocked with Blocking Solution containing RPMI 1640 supplemented with 10% heat-inactivated fetal bovine serum (Tissue Culture Biologicals, Tulare Calif.), 20 mM HEPES, 1 mM sodium pyruvate, 100 U/ml penicillin, 100 μg/ml streptomycin, and 2 mM L-glutamine (e.g. all from Life Technologies, Gaithersburg Md.) for 2 h at room temperature. Freshly isolated PBMCs (5×10⁵ cells/well) were added to wells and incubated overnight at 37° C. in a humidified 5% CO₂ atmosphere with medium, PHA, or BeSO₄. ELISpot assays were performed in triplicate. The wells were washed, and IFN-γ or IL-2 detection mAbs (BD Biosciences Pharmingen) were added, and the proteins were visualized by successive additions of avidin-horseradish peroxidase and 3-amino-9-ethylcarbazole (AEC) substrate reagent (BD Biosciences Pharmingen). The ELISpot plates were analyzed using a for example a CTL Immunospot Analyzer (Cellular Technology Ltd., Cleveland, Ohio), and results are reported as mean±SD spot-forming units (SFU) per well minus background SFU.

Statistical Analysis

In one exemplary method, ANOVA analysis was used to determine whether there was a global difference between groups. Individual contrasts were calculated to compare group means of interest after the data were checked for overall group differences, Normalizing transformations were made in cases where the data were non-Gaussian. In one example, for comparison of IL-2 versus IFN-γ expression in CBD patients, a paired t test was used. A Receiver Operator Characteristic (ROC) analysis is a statistical approach for evaluating the performance of new quantitative assays and was used to determine the optimal threshold value for a positive beryllium-induced Th1-type cytokine response as measured by sensitivity and specificity (Prism 4, GraphPad Software, Inc.). For the ROC analysis, the beryllium-sensitized group (BeS and CBD) was defined for example as “disease positive” and the normal controls as “disease negative.” A Spearman correlation was performed to analyze the association between the frequency of beryllium-specific T cells in blood and continuous variables of CBD. A P value of <0.05 was considered statistically significant.

### Example 2

### Quantification of Beryllium-Specific, IFN-γ-Producing PBMCs

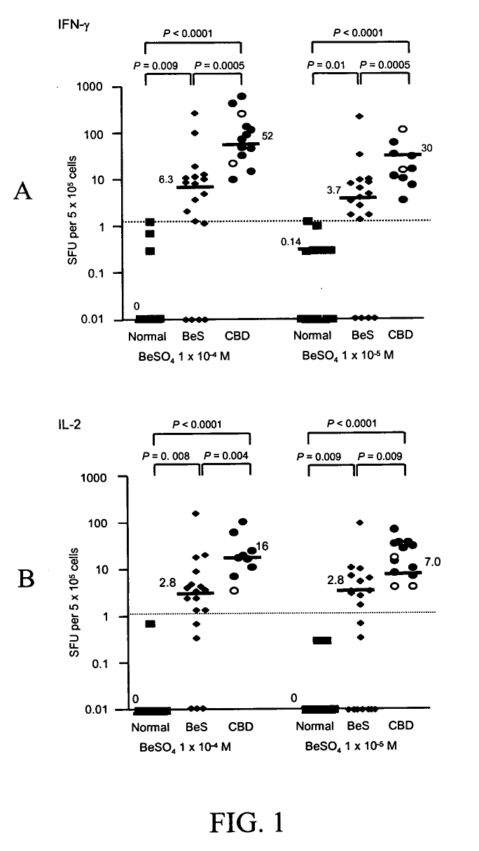

In one exemplary method ELISPOT assays were performed on fresh PBMCs from 12 healthy control, 18 BeS and 33 CBD subjects (Table 1). In response to 1×10⁻⁴ M BeSO₄, the median number of IFN-γ producing cells in blood was significantly higher in the CBD patients (52 SFU; range, 0 to 645) compared to either BeS (6.3 SFU; range, 0 to 262; P=0.0005) or normal control subjects (0.4 SFU; P<0.0001) (FIG. 1A), with similar findings seen at the lower concentration of beryllium. Within the CBD subjects, the treated individuals (shown as open triangles in FIG. 1) showed a trend towards an increased number of IFN-γ producing cells (median, 117 (range, 15-599)) in blood compared to untreated subjects (median, 34 (range, 0-645; P=0.07)). Removal of the treated subjects from the analysis did not alter the significance of differences between the groups. BeS subjects also possessed significantly greater numbers of IFN-γ-secreting cells compared to normal control subjects (P=0.009 for 1×10⁻⁴ M BeSO₄ and P=0.01 for 1×10⁻⁵ M BeSO₄).

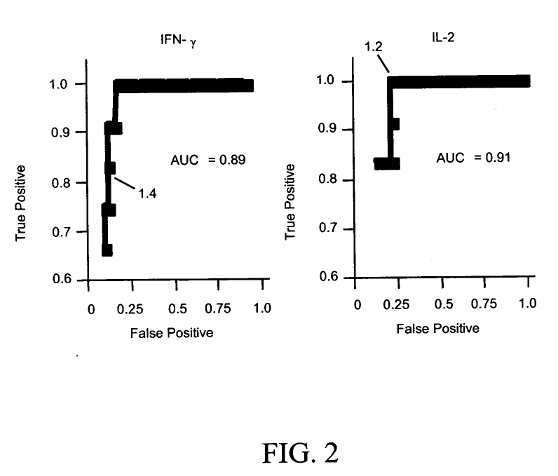

In one example, using a Receiver Operator Characteristic (ROC) curve to distinguish normal control subjects from BeS and CBD subjects, a threshold of >1.4 IFN-γ-SFU as an abnormal response was chosen (FIG. 2). With this cut-point, IFN-γ ELISPOT had a sensitivity of 80% and a specificity of 92%. Increasing or decreasing the threshold value resulted in a respective decrease in either the specificity or sensitivity. After stimulation with 1×10⁻⁴ M BeSO₄, only one of 12 healthy control subjects had a positive response, with 2.0±2.1 (mean±SD) SFU per 5×10⁵ cells. On the other hand, 13 of 18 (72%) BeS and 28 of 33 (85%) CBD subjects demonstrated beryllium-induced IFN-γ production. In one example, a ROC curve was used in order to differentiate CBD patients from BeS subjects based on the absolute number of beryllium-specific T cells in blood, a threshold value of >14 SFU for IFN-γ was chosen and had a sensitivity and specificity of 78% and 88%, respectively. As shown in FIG. 1A, only 3 of 18 BeS subjects had >14 IFN-γ SFU while 7 of 33 CBD patients were below the threshold value. Thus, in one embodiment of the present invention, differentiating CBD from BeS may be assessed based on the absolute number of beryllium-specific T cells in blood. In addition, the methods disclosed herein appear to provide a method to follow the progression of beryllium-induced disease. These methods may be useful for diagnosis, prognosis and treatment purposes for subjects suffering from beryllium-induced disease. The skilled artisan will realize the threshold value and/or other specific details of a given protocol may be further optimized to improve the specificity and/or sensitivity of the staging analysis within the scope of the claimed methods and compositions.

### Example 3

### Quantification of Beryllium-Specific, IL-2-Producing PBMCs

In one exemplary method, the median number of IL-2-producing cells was significantly higher in CBD patients (16 SFU/5×10⁵ cells; range, 0 to 226) compared to either BeS (2.8 SFU; range, 0 to 162; P=0.004) or normal control subjects (0 SFU; range, 0-1; P<0.0001) (FIG. 1B). Similar findings were observed at the lower concentration of beryllium. BeS subjects also possessed significantly greater numbers of IL-2 secreting cells compared to normal control subjects (P=0.008 for 1×10⁻⁴ M BeSO₄ and P=0.009 for 1×10⁻⁵ M BeSO₄).

An exemplary ROC analysis was used to determine the threshold for a positive beryllium induced IL-2 response (FIG. 2). With a cut-point of >1.2 IL-2-SFUs, IL-2 ELISPOT had a sensitivity of 78% and a specificity of 100%. Due to the lower number of IL-2-secreting T cells in the blood of CBD patients, more overlap was observed between the number of IL-2-producing, beryllium-specific T cells in BeS and CBD subjects. In one exemplary method, a ROC analysis was used to differentiate CBD patients from BeS subjects based on the absolute number of circulating IL-2-producing, beryllium-specific T cells, a cutoff value of >9.2 SFUs had a sensitivity and specificity of 66% and 83%, respectively.

### Example 4

### Proliferation of PBMCs from BeS and CBD patients to BeSO₄

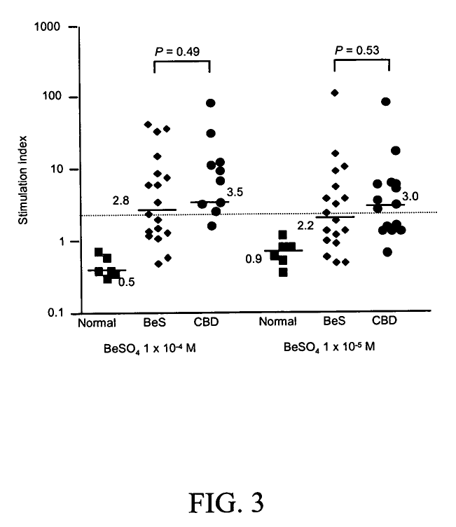

In another exemplary method, PBMCs from healthy control, BeS, and CBD subjects described above were examined for beryllium-induced proliferation at the same time as the ELISPOT assays were performed. As shown in FIG. 3, none of the control subjects demonstrated a beryllium-induced proliferative response, with a median SI of 0.5 (range, 0.2-1.4) for 1×10⁻⁴ M BeSO₄ (one example of a beryllium composition) and 0.7 (range, 0.4-1.4) for 1×10⁻⁵ M BeSO₄. All of the BeS and CBD patients enrolled in this study had a positive proliferative response at some point during their course. No significant difference in beryllium-induced proliferation of PBMCs from CBD versus BeS subjects was seen for either BeSO₄ concentration (FIG. 3). The median SI for PBMCs from BeS and CBD patients exposed to 1×10⁻⁴ M BeSO₄ was 2.8 (range, 0.5-42) and 3.5 (0.6-85) (P=0.49), respectively. The findings were similar when expressed as ΔCPM, with the median ΔCPM from BeS and CBD patients exposed to 1×10⁻⁴ M BeSO₄ being 917 (range, 0-36,355) and 1514 (range, 0-20,884), respectively (P=0.85). Overall, 10 of 18 (55%) BeS patients exhibited a positive proliferative response to 1×10⁻⁴ M BeSO₄ compared to 23 of 33 (70%) CBD patients. Similar findings were seen with 1×10⁻⁵ M BeSO₄. In one exemplary method, ELISPOT analysis for IFN-γ was able to detect the presence of beryllium-specific T cells in blood. For example, 33 of 51 (65%) CBD and BeS subjects using BeLPT had a positive beryllium-induced proliferative response in this study compared to 41 of 51 (80%) patients with a positive ELISPOT assay (X²=8.6; P=0.003). In 6 BeS and 7 CBD subjects, the ELISPOT assay detected IFN-γ- and/or IL-2-secreting cells in response to beryllium exposure in culture while no beryllium-induced proliferation was detected at the time of the study. Compared to BeS and CBD patients with a positive blood BeLPT, similar numbers of IFN-γ- and IL-2-expressing cells were seen in the blood of subjects with a negative BeLPT. For example, in CBD patients, the median number of beryllium-induced, IFN-γ-expressing cells was 83 SFU (range, 0-645) in subjects with and 48 SFU (range, 10-112; P=0.3) in subjects without beryllium-induced proliferation. Beryllium-induced proliferation occurred in the absence of detectable cytokine secretion in only 2 CBD patients, while both ELISPOT analysis and proliferation assay were negative in 2 BeS and 3 CBD patients. These results demonstrate the advantages of the present methods, compared to standard current techniques of screening for beryllium-induced disease.

### Example 5

### Comparison of IFN-γ Versus IL-2 Expression on CD4+ T Cells Following BeSO4 Exposure in Culture

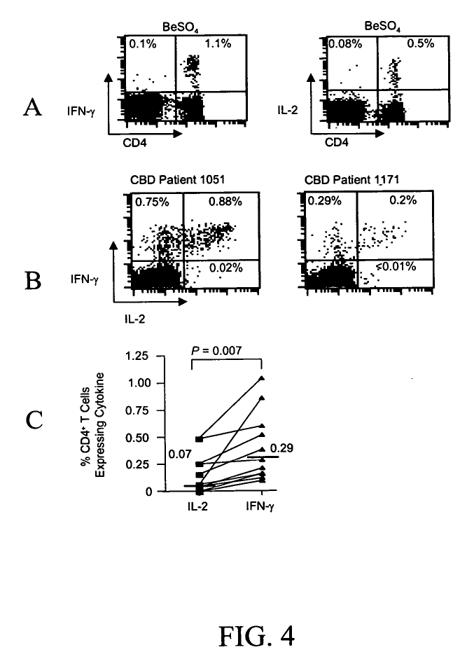

In one exemplary method, ELISPOT analysis of blood cells suggested that a higher percentage of beryllium-specific cells from CBD patients selectively produced IFN-γ (or lost the ability to secrete IL-2) compared to cells from BeS subjects. For example, the ratio of IFN-γ- to IL-2-secreting cells determined by ELISPOT analysis was 2.7 (median; range, 0.5-167) for CBD patients (n=33) and 1.8 (median; range, 0.5-6.9) for BeS patients (n=18) (P=0.07). To further address this issue, intracellular cytokine staining was performed on blood from 11 of the 33 CBD subjects who had large enough populations (>0.04%) of circulating beryllium-specific CD4+ T cells to allow for evaluation of Th1-type cytokine expression after beryllium exposure in culture. As shown in FIG. 4A, 1.1% and 0.5% of the CD4+ T cells from this representative subject expressed IFN-γ and IL-2, respectively, following beryllium exposure. IFN-γ and IL-2 expression in CD4− T cells was equal to background levels. In the peripheral blood, the beryllium-specific CD4+ T cells appeared equally divided into two groups: T cells capable of expressing both IFN-γ and IL 2 and another expressing only IFN-γ (FIG. 4B). Similar to T cells in the lung (12), almost no cells were detected that produced IL-2 in the absence of IFN-γ expression. In these eleven CBD patients, the median percentage of IFN-γ-expressing CD4+ T cells was 0.29% (range, 0.1%-1.1%) compared to 0.07% for IL-2 (0%-0.49%; P=0.007) (FIG. 4C).

### Example 6

### Analysis of Blood Beryllium-Specific CD4+ T Cells in Relation to Clinical Assessment of BeS and CBD Patients

In one exemplary method, the relationship was evaluated between the frequency of IFN-γ- (or IL-2-) secreting T cells in blood and various markers of disease severity (Table 2). No correlation was seen between the frequency of beryllium-induced-IFN-γ expression in blood T cells and the duration of beryllium exposure in the workplace (r=0.02; P=0.89) or duration of disease diagnosis (r=0.26; P=0.15). High numbers of beryllium-specific cells in blood were associated with the extent of alveolar inflammation as measured by the BAL total WBC count (r=0.41; P=0.004) and BAL lymphocyte count (r=0.45; P=0.002). These findings suggest that IFN-γ ELISPOT analysis may be used to obtain a glimpse into the target organ without the need for invasive procedures. It is contemplated herein that beryllium-induced IFN-γ or IL-2 production in blood T cells may be used to assess the level or severity of alveolar inflammation in a subject having beryllium-induced disease.

All of the COMPOSITIONS and METHODS disclosed and claimed herein can be made and executed without undue experimentation in light of the present disclosure. While the systems, compositions and methods of this invention have been described in terms of preferred embodiments, it will be apparent to those of skill in the art that variations may be applied to the COMPOSITIONS and METHODS and in the steps or in the sequence of steps of the methods described herein without departing from the concept, spirit and scope of the invention. More specifically, it will be apparent that certain agents that are both chemically and functionally related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of detecting beryllium-induced disease in a subject comprising:
a) obtaining a sample comprising CD4+ T cells from a subject;
b) exposing the cells to a beryllium composition; and
c) measuring Th1-type cytokine production from the cells;
wherein Th1-type cytokine production in response to beryllium indicates the presence of a beryllium-induced disease.

2. The method of claim 1, wherein the beryllium-induced disease is beryllium sensitivity (BeS) or chronic beryllium disease (CBD).

3. The method of claim 1, wherein measuring Th1-type cytokine production is measuring Th1-type cytokine production using an ELISPOT assay.

4. The method of claim 1, wherein measuring Th1-type cytokine production from the cells is selected from the group consisting of measuring IFN-γ, measuring IL-2, and measuring IFN-γ plus IL-2.

5. The method of claim 1, further comprising selecting a cut-point for Th1-type cytokine production in response to beryllium.

6. The method of claim 5, further comprising differentiating cut-points in subjects with BeS and subjects with CBD.

7. The method of claim 6, further comprising monitoring the progression of beryllium-induced disease from BeS to CBD.

8. The method of claim 1, wherein obtaining a sample is selected from the group consisting of obtaining a sample of peripheral blood, obtaining a sample of an enriched white cell fraction of blood and obtaining a sample of bronchoalveolar lavage.

9. A kit for a subject having or at risk of developing beryllium disease comprising:
at least one container to hold a sample from a subject;
a first agent delivered to the container wherein the first agent comprises a beryllium composition capable of inducing the production of Th-1 type cytokines;
at least one reagent to measure Th-1 type cytokine produced;
and at least one internal control.

10. The kit of claim 9, wherein the Th-1 type cytokine is IL-2.

11. The kit of claim 9, wherein the Th-1 type cytokine is IFN-γ.

12. A method for assessing a treatment for beryllium-induced disease in a subject comprising:
a) obtaining a sample comprising CD4+ T cells from a subject undergoing treatment for beryllium-induced disease;
b) exposing the cells to a beryllium composition; and
c) measuring Th1-type cytokine production from the cells;
wherein the Th1-type cytokine production from the cells in response to beryllium indicates a stage of progression of beryllium-induced disease.

13. The method of claim 12, wherein measuring Th1-type cytokine production is measuring Th1-type cytokine production using an ELISPOT assay.

14. The method of claim 12, wherein measuring Th1-type cytokine production from the cells is selected from the group consisting of measuring IFN-γ, measuring IL-2, and measuring IFN-γ plus IL-2.

15. The method of claim 1, further comprising selecting a cut-point for Th1-type cytokine production in response to beryllium.
